(19) United States
(12) Patent Application Publication
(10) Pub. No.: US 2004/0047807 A1
(43) Pub. Date: Mar. 11, 2004

(54) Use of neurotoxic substances in producing a medicament for treating joint pains

(76) Inventor: Dominik Meyer, Zurich (CH)

Correspondence Address: RANKIN, HILL, PORTER & CLARK, LLP, 700 HUNTINGTON BUILDING, 925 EUCLID AVENUE, SUITE 700, CLEVELAND, OH, 44115-1405, US

(21) Appl. No.: 10/466,973

(22) PCT Filed: Jan. 24, 2001
(86) PCT No.: PCT/CH01/00053

(51) Int. Cl. A61K 49/04; A61K 31/05

(52) U.S. Cl. 424/9.42; 514/737

(57) ABSTRACT

The present invention relates to the use of neurotoxic substances, which have a toxic effect in particular for the axon and the nociceptive nerve endings, for the preparation of an agent for the treatment of joint pain.

The present invention relates to the use of neurotoxic substances for the preparation of an agent for treating joint pain according to the preamble of claim 1 and a method for applying this agent into the intracapsular space or into the synovial sac of the joint according to the preamble of claim 30.

Pain emanating from joints often originates in the area of the joint capsule or in the bone area close to the joint. This may involve many etiologies such as, for instance, arthrotic or arthritic diseases, mechanical or other irritation of the bone surface near the joint, infections, autoimmune processes, etc. In all cases of interest for the purpose of the present invention, the developing pain emanates from nociceptive nerve fibers in the area near the joint. Nociceptive nerve fibers are also called C-fibers and A-delta fibers. If an analgesic substance (such as local anesthetics of morphine) is injected into such a diseased joint, the patient's symptoms are alleviated. However, the effect of the most common substances today is of only limited duration, and the pain usually recurs.

Today, the following procedures are generally used for treating painfully-diseased joints:

Physiotherapy/motion therapy

Systemic analgesic/antiinflammatory therapy (etc.)

Local analgesic/antiinflammatory procedures (etc.)

Surgical procedures

Arthroscopic: debridement, joint cleaning, etc.

Open/mini-open: joint replacement, joint stiffening, etc.

In the literature, a series of known substances for treating painful, inflammatory joints have been recommended, in particular:

Osmic acid or radioactive substances such as technetium 99, which resulted in synoviorthesis;

Injection of local anesthetics, hyaluronic acid preparations (etc.)

Injection of antiinflammatories

Injection of contrast agents for joint diagnostics

Joint flushing for cleansing joint

Chemical, thermal, electrical or surgical ablation of the nerves supplying the joint.

All substances and procedures used until now lead to only a relatively short or incomplete freedom from pain, or cause permanent damage to the joint.

For instance, the known synoviorthesis method has the disadvantage of denaturing the structures, in particular the proteins, which act as inflammation triggers in the developmental process of arthritis and, in part, of arthrosis. This creates a fibrosis of the joint capsule that is less inflammatory and therefore less painful. At the same time, the fibrosis occurring during synoviorthesis of the joint reduces the usually-present hyperemia, which also needs to be treated, resulting in a therapeutic benefit as well. The fibrotic post-synoviorthesis scarring, however, may also lead to reduced mobility of the joint, as well as to reduced production of synovial fluid. This undesired fibrosis of the joint capsule should be prevented, and only the sensory innervation of the joint should be eliminated.

This is where the present invention comes in. The object of the invention is to search for suitable substances and to develop a method for injecting such substances, which permanently damage the nerve ends responsible for nociception for long-term analgesia, without endangering structures distant from the joint.

The present invention achieves the object by using neurotoxic substances according to claim 1 and a method according to the characteristics of claim 30.

In the following, the invention is described as it applies to humans; in particular, the doses given relate to human application. The invention, however, is also suitable for the veterinary sector, where adaptations in dosage must be made as a function of the body weight of the respective animal.

Phenol and phenol derivatives, including analogs and their pharmacologically acceptable salts, have proven to be particularly suitable substances for producing an agent for treating joint pain. Among the phenol derivatives, cresols, above all, have proven effective, in particular ortho, meta and para cresols and their derivatives. Of the cresol derivatives, mainly chloro cresols are suitable, in particular 2-chloro-m-cresol, 3-chloro-p-cresol, 4-chloro-m-cresol, 3-chloro-o-cresol, 6-chloro-o-cresol, 2-chloro-pcresol, 5-chloro-o-cresol, 6-chloro-m-cresol and 4-chloro-o-cresol. Eugenol and thymol and their derivatives haven proven effective as well.

Another preferred group of neurotoxic substances that have proven themselves are alcohols, in particular ethyl alcohol.

Another preferred group of neurotoxic substances that have proven themselves are cytostatic drugs, in particular those having neuropathic side effects. So-called tubular and spindle poisons for the disruption of the axonal transport and the reduction of the Wallerian degeneration are of particular effectiveness.

Cytostatic drugs that have proven to be particularly effective are taxanes such as paclitaxel (>200 mg/M² body surface), taxol (>200 mg/m² body surface), as well as vinca alkaloids such as vincristine (1.4 mg/M² body surface), vinblastine (6 mg/M² body surface), vindesine (3 mg/M² body surface), vinorelbine (30 mg/M² body surface), and finally also the marine cytostatic drugs aplidine, didemnin B, isohomohalichondrin B (IHB).

Another effective group of cytostatic drugs consists of alkylating substances, in particular platin complexes such as cisplatin (DDP) with 50-75 (up to 120) mg/m² body surface or 2 mg per kg body weight/week, or carboplatin (50 to 450 mgl).

Another preferred group of neurotoxic substances that have proven themselves are nitriles, preferably 1,3-butenenitrile (allyl cyanide) at a quantity of >20-40 mg/kg body weight; cis/trans-2-butenenitriles (crotononitriles) at a quantity of >50-100 mg/kg body weight; as well as 3,3′-lminodipropionitrile at a quantity of 50-100 mg/kg body weight.

Another favorite group of proven neurotoxic substances is local anesthetics. Particular effectiveness has been shown by highly-concentrated local anesthetics administered at normal doses such as lidocaine, preferably at a concentration of over 6%, max. dose of 500 mg; prilocaine, preferably at a concentration of over 3%, max. dose of 600 mg; mepivacaine, preferably at a concentration of over 5%, max. dose of 500 mg; bupivacaine, preferably at a concentration of over 1.5%, max. dose of 150 mg; levobupivacaine, preferably at a concentration of over 5%; ropivacaine, at a concentration of over 2%; etidocaine, preferably at a concentration of over 2%, max. dose of 300 mg; procain, preferably at a concentration of over 3%, max. dose of 600 mg; chloroprocaine, preferably at a concentration of over 3%, max. dose of 800 mg; tetracaine, preferably at a concentration of over 2%, max. dose of 100 mg. furthermore, lidocaine-compounds such as lidocaine (8%) and its compounds as, for instance, N-beta-phenylethyl-lidocaine at high concentration.

The total quantities of local anesthetics to be used are approximately the same as the quantities indicated for phenols and cresols.

When using local anesthetics as neurotoxic substances, acid additives have shown to increase the effectiveness, for instance NaHSO₃ added to chloroprocaine. This lowers the pH-value to approx. 3, increasing the effectiveness according to the present invention of the local anesthetic.

The above-mentioned substance groups according to the present invention have the following advantageous characteristics:

Long-time effect

May be used just once

Not toxic sytemically in effective dosage

Predominantly neurotoxic/neutolytic for sensory fibers, less so for propioceptive fibers and for motor fibers

Acts quickly

Not toxic to synovia

Not toxic to bones

Not toxic to ligaments

Not toxic to cartilage

Not toxic to blood vessels

Not painful upon injection

Not very or reversibly harmful when escaping from the joint capsule

Soluble and injectible

May be mixed with the desired additives

Recovery possible in the case of lesion of motoneurons

Does not promote inflammation

Germicidal

The method according to the present invention relates to the injection of a neurotoxic, neurolytic, neuroparalytic or long-term analgesic substance (referred to in the following and in particular in the claims overall as “neurotoxic” substances), into a painful or diseased human or animal body joint. This substance can either be left there or be completely or partially suctioned off after a certain time of action. The therapeutic substance now diffuses to the sensory nerve endings that directly or indirectly innervate the joint area, predominantly inhibits or damages them, and leads to a reduced perception of joint pain. The new aspect of this method is that the joint capsule or the joint's synovial sac is used to concentrate the effect of the therapeutic substance on the location of the origin of the pain and thereby permit a higher concentration locally of the therapeutic substance than would be possible without the protective joint capsule or the synovial sac of the joint at the same concentration and tolerance, while at the same time being relatively gentle on the vessel/nerve structures and other structures in the vicinity of the joint. This achieves a long-term alleviation of the sensation of pain emanating from the ligament-capsule-joint-complex by suppressing or eliminating the transmission of impulses. This method may be used preventively as well as therapeutically. At the same time, the disinfecting action of the neurotoxic substance kills potential infective agents, a property that may also be used therapeutically.

The advantages of the use according to the present invention of the neurotoxic substances and the method according to the present invention of their injection into the joint capsule or the synovial sac of the joint are as follows:

The intraarticular injection of selectively neurotoxic substances for the analgesic treatment of joints leads to a great relief of the capsule-ligament structures, the synovia and the cartilage-bone structures and therefore to the preservation of the physiological conditions.

The use of the joint capsule as a natural boundary to the distribution of a neurotoxic substance.

The onset of the effect of the neurotoxic substances is not dependent on specific neuronal epitopes.

The method may be performed by nonspecialists.

The method may be performed using a thin, even nonarthroscopic needle.

The method does not pose a risk of infection, in contrast to the popular cortisone-injection method, which is locally highly infection-promoting, since cortisone locally inhibits the immune system.

The method leads to a sensory denervation, i.e. a short-circuiting of pain-conducting nerves.

Broadening of the joint mobility by eliminating the painful mobility restriction, compared to synoviorthesis, where a mobility restriction occurs because of the developing capsule fibrosis.

Positive preparation for later arthroplasty. The sclerotizing effect of the neurotoxic substance (on the one hand as a result of a chemical-biological reaction, on the other hand due to the mechanical load during painfree joint usage) gives the bone near the joint a favorable structure for the later retention of a prosthesis.

No local fatty-tissue resorption (lipolysis).

No weakening of collagenous tendon/ligament/capsule structures.

In an preferred embodiment of the present invention an x-ray contrast agent such as a barium additive is used in addition to the neurotoxic substance to enable imaging monitoring of the distribution of the neurotoxic substance in the intracapsular space. The following substances may be used as contrast agents, depending on the method:

X-ray, CT: iodine-containing substances such as triiodinated benzoates or iopamidol, ideally 30-80 g/100 ml or for example, 5-10% of another contrast agent such as barium.

MRI: gadolinium, for example; for instance per 1 ml: 469.01 mg gadopentate dimeglumine, 0.99 mg meglumine, 0.4 mg diethylenetriaminepentaacetate.

In another preferred embodiment of the present invention, an antibiotic, disinfecting and/or sterilizing substance is added in addition to the neurotoxic substance.

In another preferred embodiment of the present invention, a viscous additive such as hyaluronic acid, preferably at a concentration of 0.1-10.0 mg/ml injection solution, is used in addition to the neurotoxic substance, which leads to a mechanical glide improvement of the joint.

In another preferred embodiment of the present invention, a vasoconstrictor, preferably adrenalin, noradrenalin or other, similar, preferably alpha-adrenergic vasoconstrictors are used in addition to the neurotoxic substance. With adrenalin, the total dose of the neurotoxin (i.e. the substance toxic to the peripheral nervous system) may be increased by a factor of 2, since the systemic effect is reduced due to the decreased resorption. The adrenalin concentration may be (1:10,000 to) 1:80,000 to 1:200,000. The total adrenalin dose is <0.25 mg. A 50-ml solution of 1:200,000 adrenalin contains 0.25 mg adrenalin.

In another preferred embodiment of the present invention, an antiphlogistically-acting substance, for instance nonsteroidal antirheumatic agents such as COX-2 inhibitors, acetylsalycylic acid, etc., is used in addition to the neurotoxic substance.

In another preferred embodiment of the present invention, a steroid is used in addition to the neurotoxic substance in order to be able to control a possibly-occurring inflammatory reaction. Furthermore, this makes it possible to add a rather causal treatment of painful, inflammatory joint diseases, supporting the symptomatic, neurolytic treatment. Betamethasone has proven to be especially suitable; for instance, 5 mg betamethasone in the form of dipropionate (crystalline suspension) and betamethasone in the form of disodium phosphate (solution in 1 ml, may be added to the total quantity to be injected). This solution is equivalent to 45/23 mg prednisone/prednisolone.

In another preferred embodiment of the present invention, glycerol is used as a solvent in addition to the neurotoxic substance. Glycerol also has neurotoxic properties (but in particular if it is injected intraneurally). In addition, glycerol acts as a joint lubricant, so that it also has a physical effect. The glycerol concentration is preferably between 10% and 95%.

In another preferred embodiment of the present invention, an analgesic is used in addition to the neurotoxic substance in order to achieve short-term analgesia in the event that the neurolytic effect is delayed and there is an initial painful period. Highly-concentrated, but normally-dosed, local anesthetics have proven to be particularly effective, for instance the above-mentioned substances.

Water, salt solution, sodium iothalamate, iophendylate, ricin, polyethylene glycol or propylene glycol may also be used as a solvent medium in place of water. The advantage of glycerol as a diluting solution is that it is hyperbaric and somewhat neurotoxic itself.

Some agents have proven to increase the effectiveness of neurotoxic substances, as for instance antioxidants, preservatives and excipients, in particular sodium bisulfite (>0.2%), NaHSO₃, ammonium compounds such as ammonium sulfate (NH₄)₂SO₄, 2-10 (−30%), polysorbate 80 (PS80) 0.025 mg/ml.

The neurotoxic substance is preferably dissolved in a solvent well tolerated by the body and is expediently injected in a volume corresponding to the available space in the joint to be treated, so that the latter is completely filled. This achieves the advantage of an optimum distribution of the neurolytic substance. However, it is also possible to inject less fluid, in which case the joint must be moved well to better distribute the neurolytic substance.

The volume of fluid to be injected into the intracapsular area may range from 0.1 to 150 ml. For a finger joint a maximum of approximately 1 ml is enough; for the shoulder joint, a maximum of 10 ml; for the knee joint, approximately 30-50 ml.

The dosage of the neurolytic substance depends on its absolute solubility in the selected solvent medium. The capsule thickness of the involved joint has a significant effect on the dosage. The thicker the capsule, the more neurolytic substance is required.

When using chlorocresol as a neurolytic substance in glycerol as a solvent well tolerated by the body, a quantity ratio of chlorocresol : glycerol of 1:5 to 1:70, preferably 1:40 to 1:50 should be selected.

When using phenol in glycerol, a concentration range of 0.5%-40.0%, preferably 3%-12%, should be selected.

In order to better clarify the present invention, several examples of advantageous embodiments are described below.

### EXAMPLE 1

After a facultative, diagnostic injection of local anesthetics—either immediately or some time in advance—the therapist placed an injection needle in the joint cavity of a knee joint and injected 40 ml of a solution of m-chlorocresol in glycerol into the intracapsular space under facultative, simultaneous (image converter, CT, sonography, MRI, etc.) or subsequent (x-ray, CT, MRI, sonography, etc.) imaging monitoring. The patient experienced significant relief after as early as 14 hours after the intervention. This lasted for over six months.

### EXAMPLE 2

The injected solution corresponded to that of Example 1, except that for the imaging method to be used 5 ml of a visible contrast agent (iopamidol at a concentration of 50 g/100 ml) was added, which spread after injection inside the joint capsule and documented the position of the injection needle and the distribution of the therapeutic substance within the capsule. The neurotoxic substance in the injected solution was suctioned off immediately after the injection was made. However, it may also be suctioned off after a defined, substance-dependent time of action or not at all. The patient experienced significant relief after as early as 15 hours after the intervention. This lasted for over eight months.

### EXAMPLE 3

The therapist placed a thin infusion catheter, analog to an epidural catheter, in the joint in question and used a perfusor [syringe pump] to inject a neurotoxic substance of low concentration in this case (2-5% chlorocresol, 5% hydrocortisone (facultative), 80-95% glycerol, 0-10% contrast agent) into the joint in question at a rate of 1-10 ml/h during 12 hours. He also facultatively placed a drainage catheter having a facultatively-defined outflow resistance (e.g. 20 mmHg), in order to achieve a fluid turnover. This method allowed the therapist to achieve uniform infiltration of the painful joint, without large concentration peaks. Furthermore, the time of action could be better defined. During a subsequent arthroscopy after 1, 2, 7, 14 and 28 d it could be demonstrated that only small amounts of inflammatory tissue were present. The patient experienced significant relief after as early as 12 hours after the intervention. This lasted for over one year.

### EXAMPLE 4

After implantation of an artificial joint (e.g. knee), the therapist injected 50 ml of the neurotoxic substance into the reclosed joint capsule (in another embodiment: into the periprosthetic area without capsule). This minimized the postoperative pains. In this case, too, the neurotoxic substance had a low concentration (5chlorocresol in glycerol as solvent), in order to facilitate a subsequent reinnervation.

### EXAMPLE 5

In the case of a patient with painful septic loosening of a total hip endoprosthesis, the neurotoxic substance (here highly concentrated: 5% chorocresol in glycerol) could be injected into the (neo)capsule around the prosthesis, causing the patient to experience a long-term relief from pain (over one year) within a few (6-12) hours. In addition, the infection around the prosthesis could be greatly contained along the shaft of the prosthesis and around the socket, and even completely eliminated in some cases, by the diffusion of the neurotoxic substance (which also had an antiseptic effect). Facultatively, this treatment may be aided by systemically-administered antibiotics (such as rifampicin 450 mg, ciprofloxacin 750 mg).

Radiologically, a consolidation of the bone substance could be demonstrated around the prosthesis.

### EXAMPLE 6

In the case of a patient with painful capsulitis of joints (e.g. “frozen shoulder”), the neurotoxic substance was injected into the joint. Again, the distribution of the substance could be monitored by imaging with the addition of the appropriate contrast agents. Facultatively, an antiphlogistically-acting substance was admixed. A few minutes after the injection, the pain subsided permanently, so that the patient regained the mobility lost due to capsulitis. With this treatment, sometimes just a temporary analgesia (2-3 weeks) is desired, which is why here the concentration of the neurotoxic substance was kept rather low (2-3% chlorocresol).

### EXAMPLE 7

The therapist injected 5 ml of a neurotoxic substance consisting of 8% phenol and 5% cortisone in glycerol as solvent into a chronically-inflamed trochanteric bursa via the greater trochanter of the hip.

Within 60 minutes, the complaints of the patient disappeared, and the patient remained painfree at this location for several years.

### EXAMPLE 8

The therapist injected 1 ml of a neurotoxic substance consisting of 15% lidocaine, adrenalin (1:80000), as well as 5% contrast agent in a physiological salt solution as solvent into a painful, arthrotic finger joint. After approximately 15 minutes the complaints of the patient disappeared for several months. The correct position of the injection needle could be documented via the contrast agent.

### EXAMPLE 9

The therapist injected a mixture of 5% chlorocresol, 10% lidocaine, as well as vincristine in a quantity of 0.7 mg/m² body surface in glycerol as solvent. This mixture showed a particularly lasting effect, since its components damage the intended nerves in different ways. The effect of chlorocresol lies in the fact that it dissolves the nerve membrane; that of lidocaine in that it destroys the nerves via irreversible receptor blocking, as well as via toxic intracellular Ca-release; and that of vincristine in that it permanently prevents nerve regeneration and inhibits the axonal transport.

In a variant, the mixture additionally contained adrenalin 1:80000 and 10% contrast agent as inactive ingredients.

All variants of this mixture proved especially effective for permanent nerve destruction.

1. Use of neurotoxic substances belonging to the group of cresols and their derivatives for the preparation of an agent for the treatment of joint pain.

2. Use according to claim 1, wherein the neurotoxic substances are toxic predominantly for pain-conducting (nociceptive) nerve fibers.

3. Use according to claim 1 or 2, wherein the neurotoxic substances are selected from that group which is toxic for the axon and the nociceptive nerve endings.

4. Use according to one of claims 1 through 3, wherein the neurotoxic substances are less neurotoxic for motor and propioceptive nerve fibers than for sensory nerve fibers.

5. Use according to one of claims 1 through 4, wherein the neurotoxic substances belong to the group of ortho-, meta- and para-cresols and their derivatives.

6. Use according to claim 6, wherein the cresol derivatives comprise chlorocresols, in particular 2-chloro-m-cresol, 3-chloro-p-cresol, 4-chloro-m-cresol, 3-chloro-o-cresol, 6-chloro-o-cresol, 2-chloro-p-cresol, 5-chloro-o-cresol, 6-chloro-m-cresol and 4-chloro-o-cresol.

7. Use according to one of claims 1 through 6, wherein, in addition to the neurotoxic substances, an x-ray contrast agent is used, preferably gadolinium-containing, iodine-containing or barium-containing substances.

8. Use according to one of claims 1 through 7, wherein, in addition to the neurotoxic substances, glycerol is used, preferably at a concentration of 10 percent by weight to 95 percent by weight.

9. Use according to one of claims 1 through 8, wherein, in addition to the neurotoxic substances, steroids are used.

10. Use according to one of claims 1 through 9, wherein, in addition to the neurotoxic substances, a vasoconstrictor is used, preferably adrenalin, noradrenalin, phenylephrin or ornipressin.

11. Use according to one of claims 1 through 10, wherein the neurotoxic substances are dissolved in a solvent well tolerated by the body, preferably glycerol, iophendylate or propylene glycol.

12. Use according to one of claims 1 through 11, wherein the neurotoxic substances are used for denervation in the degeneratively-diseased joints.

13. Method for the treatment of joint pain, wherein a neurotoxic substance belonging to the group of cresols and their derivatives is injected into the intracapsular area or into the synovial sac of the joint affected by pain.

14. Method for the treatment of joint pain according to claim 13, wherein the neurotoxic substance is dissolved in a solvent well tolerated by the body, and a fluid volume of 0.1 to 150 ml is injected into the intracapsular area or into the synovial sac of the joint affected by pain.

15. Method according to claim 13 or 14, wherein the nociceptive nerve fibers are rendered impervious to pain for at least 14 days by the neurotoxic substance.
